(19) United States
(12) Patent Application Publication
(10) Pub. No.: US 2014/0056893 A1
(43) Pub. Date: Feb. 27, 2014

(54) Homodimeric Proteins

(71) Applicant: Eli Lilly and Company, Indianapolis, IN (US)

(72) Inventors: Tamer Coskun, Indianapolis, IN (US); Ryan James Darling, Fishers, IN (US); Malgorzata Donata Gonciarz, Indianapolis, IN (US); Jorge Alsina-Fernandez, Indianapolis, IN (US)

(73) Assignee: Eli Lilly and Company, Indianapolis, IN (US)

(21) Appl. No.: 13/967,396

(22) Filed: Aug. 15, 2013

Related U.S. Application Data
(60) Provisional application No. 61/777,149, filed on Mar. 12, 2013.
(60) Provisional application No. 61/691,905, filed on Aug. 22, 2012.

(51) Int. Cl. C07K 14/605; C07K 14/50

(52) U.S. Cl. CPC C07K 14/605; C07K 14/50
USPC 424/134.1; 530/387.3; 536/23.4; 435/328; 435/69.6

(57) ABSTRACT

This present invention relates to a homodimeric protein comprising fibroblast growth factor 21 (FGF21) and glucagon-like peptide (GLP-1), pharmaceutical compositions comprising the homodimeric protein, and methods for treating type 2 diabetes, obesity, dyslipidemia, and/or metabolic syndrome using such homodimeric protein.

This present invention relates to a homodimeric protein comprising fibroblast growth factor 21 (FGF21) and glucagon-like peptide (GLP-1), pharmaceutical compositions comprising the homodimeric protein, and methods for treating type 2 diabetes, obesity, dyslipidemia, and/or metabolic syndrome using such homodimeric protein.

FGF21 belongs to a family of large polypeptides widely expressed in developing and adult tissues that play crucial roles in multiple physiological functions. FGF21 is a hormone that functions as an important metabolic regulator of glucose and lipid homeostasis. FGF21 promotes glucose uptake in adipocytes by up-regulating GLUT1 expression, a mechanism distinct from that of insulin. In diabetic rodents and monkeys, human FGF21 lowered fasting serum concentrations of glucose, and reduced fasting serum concentrations of triglycerides, insulin and glucagon. Furthermore, in rodent models of diet induced obesity, FGF21 administration led to cumulative body weight loss in a dose dependent manner. Thus, FGF21 has potential utility for the treatment of diabetes, obesity, dyslipidemia, and metabolic syndrome.

In addition to its beneficial effects on type 2 diabetes, GLP-1 compounds have been described for the treatment of obesity. GLP-1 induces numerous biological effects such as stimulating insulin secretion, inhibiting glucagon secretion, inhibiting gastric emptying, inhibiting gastric motility or intestinal motility, and inducing weight loss. A significant characteristic of GLP-1 is its ability to stimulate insulin secretion without the associated risk of hypoglycemia that is seen when using insulin therapy or some types of oral therapies that act by increasing insulin expression.

Although both FGF21 proteins and GLP-1 compounds have shown positive effects in treating obesity and type 2 diabetes, there is still a need for additional beneficial therapeutics for weight loss and type 2 diabetes.

Co-administration of a FGF21 protein and a GLP-1 compound requires either injections of two separate products or a single injection of a co-formulation of two different compositions. Two injections would permit flexibility of dose amount and timing, but are inconvenient to patients both for compliance and pain. A co-formulation might also provide some flexibility of dose amounts, but it is often quite challenging or impossible to find formulation conditions that permit chemical and physical stability of both compositions due to different molecular characteristics of the two different products.

Fusion proteins comprising FGF21 and GLP-1 have been described in WO2011/020319.

The present invention provides alternative therapeutics for weight loss and diabetes. The homodimeric protein of the present invention has advantageous characteristics, which include improved potency and/or improved pharmaceutical stability. In addition to improved potency, the homodimeric protein of the present invention has one or more advantageous stability characteristics that are useful for efficient manufacturing and/or formulation as a therapeutic protein, including reduced susceptibility to hydroxylation, lowered propensity to aggregate at high concentrations, and lowered levels of post-translational modifications during production in mammalian cell systems. Additionally, the homodimeric protein of the present invention is potentially useful for the treatment of type 2 diabetes, obesity, dyslipidemia, and/or metabolic syndrome.

The present invention provides a homodimeric protein wherein the amino acid sequence of each polypeptide of said protein is

(SEQ ID NO: 1)
HGEGTFTSDVSSYLEEQAAKEFIAWLVAGGGGGGGSGGGGSGGGGSESKY
GPPCPPCPAPEAAGGPSVFLFPPKPKDTLMISRTPEVTCVVVDVSCEDPE
VQFNWYVDGVEVHNAKTKPREEQFNSTYRVVSVLTVLHQDWLNGKEYKCK
VSNKGLPSSIEKTISKAKGQPREPQVYTLPPSCEEMTKNQVSLTCLVKGF
YPSDIAVEWESNGQPENNYKTTPPVLDSDGSFFLYSRLTVDKSRWQEGNV
FSCSVMHEALHNHYTQKSLSLSLGGGGGSGGGGSGGGGSAHPIPDSSPLL
QFGGQVRQRYLYTDDAQQTECHLEIREDGTVGCAADQSPESLLQLKALKP
GVIQILGVKTSRFLCQRPDGALYGSLHFDPEACSFREDLKEDGYNVYQSE
AHGLPLHLPGDKSPHRKPAPRGPARFLPLPGLPPALPEPPGILAPQPPDV
GSSDPLRLVEPSCLRSPSFE.

The present invention also provides a DNA molecule encoding a homodimeric protein of the present invention.

The present invention also provides a mammalian cell transformed with DNA molecule(s) which cell is capable of expressing a homodimeric protein of the present invention.

The present invention also provides a process for producing a homodimeric protein of the present invention, comprising cultivating the mammalian cell under conditions such that the homodimeric protein of the present invention is expressed.

The present invention also provides a homodimeric protein of the present invention produced by said process.

The present invention also provides a pharmaceutical composition comprising a homodimeric protein of the present invention and at least one pharmaceutically acceptable carrier, diluent, or excipient.

The present invention also provides a method of treating type 2 diabetes, obesity, dyslipidemia, and/or metabolic syndrome in a patient comprising administering to the patient a homodimeric protein of the present invention.

The present invention also provides a method of treating type 2 diabetes, obesity, dyslipidemia, and/or metabolic syndrome in a patient comprising administering to the patient a pharmaceutical composition of the present invention.

Furthermore, the present invention provides a homodimeric protein of the present invention for use in therapy. Preferably, the present invention provides a homodimeric protein of the present invention for use in the treatment of type 2 diabetes, obesity, dyslipidemia, and/or metabolic syndrome.

Furthermore, the present invention provides the use of a homodimeric protein of the present invention in the manufacture of a medicament for the treatment of type 2 diabetes, obesity, dyslipidemia, and/or metabolic syndrome.

The present invention also relates to polynucleotides encoding each polypeptide of the above-described homodimeric protein of the present invention.

Furthermore, the present invention provides a polynucleotide encoding a polypeptide, wherein the amino acid sequence of the polypeptide is

(SEQ ID NO: 1)
HGEGTFTSDVSSYLEEQAAKEFIAWLVAGGGGGGGSGGGGSGGGGSESKY
GPPCPPCPAPEAAGGPSVFLFPPKPKDTLMISRTPEVTCVVVDVSCEDPE
VQFNWYVDGVEVHNAKTKPREEQFNSTYRVVSVLTVLHQDWLNGKEYKCK
VSNKGLPSSIEKTISKAKGQPREPQVYTLPPSCEEMTKNQVSLTCLVKGF
YPSDIAVEWESNGQPENNYKTTPPVLDSDGSFFLYSRLTVDKSRWQEGNV
FSCSVMHEALHNHYTQKSLSLSLGGGGGSGGGGSGGGGSAHPIPDSSPLL
QFGGQVRQRYLYTDDAQQTECHLEIREDGTVGCAADQSPESLLQLKALKP
GVIQILGVKTSRFLCQRPDGALYGSLHFDPEACSFREDLKEDGYNVYQSE
AHGLPLHLPGDKSPHRKPAPRGPARFLPLPGLPPALPEPPGILAPQPPDV
GSSDPLRLVEPSCLRSPSFE.

The present invention also provides a polynucleotide encoding each polypeptide of the homodimeric protein of the present invention, wherein the nucleotide sequence is SEQ ID NO: 2.

The polynucleotides encoding the above-described homodimeric protein may be in the form of RNA or in the form of DNA, which DNA includes cDNA, and synthetic DNA. The DNA may be double-stranded or single-stranded. The coding sequences that encode the homodimeric protein of the present invention may vary as a result of the redundancy or degeneracy of the genetic code.

The polynucleotides that encode for the homodimeric protein of the present invention may include the following: only the coding sequence for the protein, the coding sequence for the protein and additional coding sequence such as a leader or secretory sequence or a pro-protein sequence; the coding sequence for the protein and non-coding sequence, such as introns or non-coding sequence 5′ and/or 3′ of the coding sequence for the protein. Thus the term “polynucleotide encoding a protein” encompasses a polynucleotide that may include not only coding sequence for the protein but also a polynucleotide that includes additional coding and/or non-coding sequence.

The polynucleotides of the present invention will be expressed in a host cell after the sequences have been operably linked to an expression control sequence. The expression vectors are typically replicable in the host organisms either as episomes or as an integral part of the host chromosomal DNA. Commonly, expression vectors will contain selection markers, e.g., tetracycline, neomycin, and dihydrofolate reductase, to permit detection of those cells transformed with the desired DNA sequences.

The homodimeric protein of the present invention may readily be produced in mammalian cells such as CHO, NSO, HEK293 or COS cells; in bacterial cells such as E. coli, Bacillus subtilis, or Pseudomonas fluorescence; or in fungal or yeast cells. The host cells are cultured using techniques well known in the art. The preferred mammalian host cell is the CHOK1SV cell line containing a glutamine synthetase (GS) expression system (see U.S. Pat. No. 5,122,464).

The vectors containing the polynucleotide sequences of interest (e.g., the proteins of FGF21 and GLP-1, and expression control sequences) can be transferred into the host cell by well-known methods, which vary depending on the type of cellular host. For example, calcium chloride transformation is commonly utilized for prokaryotic cells, whereas calcium phosphate treatment or electroporation may be used for other cellular hosts.

Various methods of protein purification may be employed and such methods are known in the art and described, for example, in Deutscher, Methods in Enzymology 182: 83-89 (1990) and Scopes, Protein Purification: Principles and Practice, 3rd Edition, Springer, NY (1994).

The present invention also provides a process for producing a homodimeric protein, wherein the amino acid sequence of each polypeptide of said protein is SEQ ID NO: 1, said process comprising the steps of:

i) cultivating a mammalian host cell comprising a polynucleotide encoding the polypeptide having the amino acid sequence of SEQ ID NO: 1 under conditions such that said polypeptide sequence is expressed; and

ii) recovering from said host cell a homodimeric protein, wherein the amino acid sequence of each polypeptide of said protein is SEQ ID NO: 1.

The present invention also provides a homodimeric protein produced by the above-described process.

The polypeptide having the amino acid sequence given by SEQ ID NO: 5 is a homodimer when expressed in mammalian cells. “Homodimeric protein” or “homodimer”, as used herein, refers to a protein composed of two polypeptides having the same amino acid sequence wherein each polypeptide is associated with the other through non-covalent interactions and/or intermolecular disulfide bonds. Each of the polypeptides of the homodimeric protein of the present invention comprises an Fc portion (amino acids 47 to 274 of SEQ ID NO: 1). As one of skill in the art will appreciate, mammalian cell expression of such polypeptides may result in glycosylation sites of the Fc portion (amino acids 47 to 274 of SEQ ID NO: 1) at a highly conserved N-glycosylation site.

The pharmaceutical compositions of the homodimeric protein of the present invention may be administered by any means known in the art that achieve the generally intended purpose to treat type 2 diabetes, obesity, dyslipidemia, and/or metabolic syndrome. The preferred route of administration is parenteral. The dosage administered will be dependent upon the age, health, and weight of the recipient, kind of concurrent treatment, if any, frequency of treatment, and the nature of the effect desired. Typical dosage levels can be optimized using standard clinical techniques and will be dependent on the mode of administration and the condition of the patient and can be determined by a person having ordinary skill in the art.

The homodimeric protein of the present invention is formulated according to known methods to prepare pharmaceutically useful compositions. A desired formulation is a stable lyophilized product that is reconstituted with an appropriate diluent or an aqueous solution of high purity with optional pharmaceutically acceptable carriers, preservatives, excipients or stabilizers [Remington, The Science and Practice of Pharmacy, 19th edition, Gennaro, ed., Mack Publishing Co., Easton, Pa. 19951].

The homodimeric protein of the present invention may be formulated with a pharmaceutically acceptable buffer, and the pH adjusted to provide acceptable stability, and a pH acceptable for administration. Moreover, the compositions of the present invention may be placed into a container such as a vial, a cartridge, a pen delivery device, a syringe, intravenous administration tubing or an intravenous administration bag, wherein the container is a unit dose container.

The term “dyslipidemia” means a disorder of lipoprotein metabolism, including lipoprotein overproduction or deficiency. Dyslipidemia may be manifested by elevation of the total cholesterol, low-density lipoprotein (LDL) cholesterol and the triglyceride concentrations, and/or a decrease in high-density lipoprotein (HDL) cholesterol concentration in the blood.

The term “metabolic syndrome” is characterized by a group of metabolic risk factors in one person. They include: abdominal fat—in most men, a 40-inch waist or greater; high blood sugar—at least 110 milligrams per deciliter (mg/dl) after fasting; high triglycerides—at least 150 mg/dL in the bloodstream; low HDL—less than 40 mg/dl; and/or, blood pressure of 130/85 or higher.

The term “obesity” is defined as a condition in which there is an excess of subcutaneous fat in proportion to lean body mass (Stedman's Medical Dictionary 28th edition, 2006, Lippincott Williams & Wilkins).

A “patient” is a mammal, preferably a human.

The term “treating” (or “treat” or “treatment”) means slowing, reducing, or reversing the progression or severity of a symptom, disorder, condition, or disease.

The term “therapeutically effective amount” refers to the amount or dose of a homodimeric protein of the present invention which, upon single or multiple dose administration to a patient, provides the desired treatment.

The term “type 2 diabetes” is characterized by excess glucose production in spite of the availability of insulin, and circulating glucose levels remain excessively high as a result of inadequate glucose clearance.

The present invention may be practiced by referencing the following examples. However, this is not to be interpreted as limiting the scope of the present invention.

### EXAMPLE 1

### Expression of the Homodimeric Protein in CHOK1SV Cells

The homodimeric protein of the present invention is produced in a mammalian cell expression system using CHOK1SV cells. Genes coding for the homodimeric protein of the present invention are sub-cloned into the Glutamine Synthetase (GS)-containing expression plasmid backbones (pEE12.4-based plasmids). The gene encoding the homodimeric protein is constructed by ligating DNA encoding a GLP-1 compound in-frame to DNA encoding the IgG4 Fc-FGF21 protein. The cDNA sequence encoding the homodimeric protein of the present invention is ligated in frame with the coding sequence of preferred signal peptide sequences to enhance secretion of the desired product into the tissue culture medium. The preferred signal peptide sequences are the polypeptides as shown in the amino acid sequences SEQ ID NO: 3 and SEQ ID NO: 4.

The expression is driven by the viral cytomegalovirus (CMV) promoter. CHOK1SV cells are stably transfected using electroporation and the appropriate amount of recombinant expression plasmid, and the transfected cells are maintained in suspension culture, at the adequate cell density. Selection of the transfected cells is accomplished by growth in methionine sulfoximine (MSX)-containing serum-free medium and incubated at 35-37° C. and 5-7% CO₂.

Clonally-derived cell lines are generated by use of a flow cytometer. The expression of a protein in mammalian cells generally yields the GLP1 compound N-terminal sequence, HGEGT, i.e. without a methionine residue at the N-terminus, such as the homodimeric protein wherein the amino acid sequence of each polypeptide of said protein is SEQ ID NO: 1. Homodimeric proteins secreted into the media from the CHO cells are purified by Protein A affinity chromatography followed by preparative size exclusion chromatography following standard chromatographic techniques. Briefly, homodimeric proteins from harvested media are captured onto Mab Select Protein A (GE, Piscataway, N.J.) with PBS pH 7.4 running buffer; briefly washed with running buffer to remove non-specifically bound material; and eluted with 10 mM citrate pH 3.5. Fractions containing homodimeric proteins are pooled and pH is neutralized by adding 1/10 volume of 1M Tris pH 8.0. The neutralized pool is concentrated and loaded onto a Superdex 200 size exclusion chromatography column (GE, Piscataway, N.J.) with PBS pH 7.4 mobile phase. Fractions containing monomeric protein (a covalently linked homodimeric protein) are pooled, concentrated, and stored.

Alternatively, the cell free media containing homodimeric protein is treated with detergent (Triton X-100) for viral inactivation. The pH of media is adjusted to 6.0 and applied to a Capto MMC column, that is equilibrated in 10 mM citrate, 150 mM NaCl, pH 6. After sample application the resin is washed with equilibration buffer to remove non-specifically bound materials. The homodimeric protein is eluted from the column with pH gradient in 50 mM Tris, pH 8. The Capto MMC mainstream is heated to 55° C. for two hours. Precipitates that form are removed by depth filtration (Millipore). The homodimeric protein is further purified on POROS 50 HQ anion exchange column equilibrated in 50 mM Tris pH 8. Bound proteins are eluted with salt gradient in 20 mM Tris 300 mM NaCl pH 8. Eluted homodimeric protein is further purified by hydrophobic interaction chromatography. The POROS 50 HQ mainstream pool is adjusted to 1 M sodium sulfate and applied to a Phenyl Sepharose HP column equilibrated with 1 M sodium sulfate in 20 mM Tris pH 7. Homodimeric protein is eluted in a reversed salt gradient in 20 mM Tris pH 7. Purified homodimeric protein can be passed through a viral retention filter such as Planova 20N (Asahi Kasei Medical) followed by concentration/diafiltration into 10 mM citrate, 150 mM NaCl pH 7 using tangential flow ultrafiltration on a regenerated cellulose membrane (Millipore).

### EXAMPLE 2

### 3T3-L1-βKlotho Fibroblast Glucose Uptake Assay

3T3-L1-βKlotho fibroblasts are generated from 3T3-L1 fibroblasts by retroviral transduction of a CMV-driven mammalian expression vector containing the coding sequence of wild type mouse βKlotho and a blasticidin resistance marker. Blasticidin-resistant cells are selected after growth for 14 days in the presence of 15 μM blasticidin, and βKlotho protein expression is verified by immunoblot with an anti-βKlotho antibody. The 3T3-L1-βKlotho fibroblasts are maintained in Dulbecco's Modified Eagle Medium (DMEM) with 10% calf serum, and 15 μM blasticidin until plated for experimental use.

For glucose uptake, 3T3-L1-βKlotho fibroblasts are plated at 20,000 cells/well in 96-well plates and incubated for 48 hours in DMEM with 10% calf serum. The cells are incubated for 3 hours in DMEM with 0.1% bovine serum albumin (BSA) with or without the homodimeric protein of interest, followed by 1 hour incubation in Krebs-Ringer phosphate (KRP) buffer (15 mM Hepes, pH 7.4, 118 mM NaCl, 4.8 mM KCl, 1.2 mM MgSO₄, 1.3 mM CaCl₂, 1.2 mM KH₂PO₄, 0.1% BSA) containing 100 μM (glucose with or without the homodimeric protein of interest. Non-specific binding is determined by incubation of select wells in Krebs-Ringer bicarbonate/Hepes (KRBH) buffer containing 1 mM 2-deoxy-D-(¹⁴C) glucose. The reaction is terminated by addition of 20 μM cytochalasin B to the cells and glucose uptake is measured using a liquid scintillation counter.

The in vitro potency (EC₅₀) of the homodimeric protein wherein the amino acid sequence of each polypeptide of said protein is SEQ ID NO: 1 in the 3T3-L1-βKlotho fibroblast glucose uptake assay is 0.283±0.026 nM with 95% confidence interval of 0.189 to 0.423.

### EXAMPLE 3

### Human 293 cell-βKlotho-SRE luc Assay

### Construction of 293-βKlotho-SRE luc Reporter Cells:

HEK-293 (human embryonic kidney cells) are cultured at 37° C., 5% CO₂ in growth medium (GM) containing 10% fetal bovine serum (FBS) in Dulbecco's modified Eagle's medium. Cells are cotransfected with a plasmid containing a CMV promoter driven human βKlotho expression cassette and a plasmid containing a Serum Response Element (SRE) driven luciferase expression cassette. The βKlotho expression plasmid also contains an SV40 promoter driven neomycin phosphotransferase expression cassette to confer resistance to the aminoglycoside antibiotic G418. Transfected HEK-293 cells are selected with 600 μg/mL of G418 to select for cells where the transfected plasmids have been integrated into the genome. Selected cells are cloned by dilution and tested for an increase in luciferase production at 24 hours post addition of the homodimeric protein of interest. The clone demonstrating the largest FGF21 dependant increase in luciferase is chosen as the cell line used to measure relative proteins activity.

### 293-βKlotho-SRE luc FGF21 Activity Assay:

293-βKlotho-SRE luc cells are rinsed and placed into CD 293 suspension culture media (Invitrogen). Cells are grown in suspension overnight at 37° C., 6% CO₂, 125 rpm. Cells are counted, pelleted by centrifugation, and re-suspended in CD 293 media containing 0.1% BSA. Cells are placed in white 96 well plates at 25,000 cells per well. A four-fold serial dilution in CD 293/0.1% BSA is prepared for the homodimeric protein of interest to generate eight dilutions with final concentrations from 100 nM to 0.006 nM. Dilutions are added to cells in triplicate and incubated for 16-20 hours at 37° C., 5% CO₂. Luciferase level is determined by the addition of an equal volume of OneGlo™ luciferase substrate (Promega) and measuring relative luminescence. Data is analyzed using a four parameter logistic model (XLfit version 5.1) to fit the curves and determine EC₅₀.

The average in vitro potency (EC₅₀) of the homodimeric protein wherein the amino acid sequence of each polypeptide of said protein is SEQ ID NO: 1 in the human 293 cell-βKlotho-SRE luc assay is 0.283±0.038 nM.

### EXAMPLE 4

### In Vitro Glucagon-Like-Peptide 1 Receptor (hGLP-1R)-Stimulated cAMP Functional Assay

The GLP-1 stimulated cAMP functional assay cloned human glucagon-like peptide 1 receptor (hGLP-1R) (Graziano M P, Hey P J, Borkowski D, Chicchi G G, Strader C D, Biochem Biophys Res Commun. 1993 Oct. 15; 196(1):141-6) isolated from 293HEK membranes. The hGLP-1R cDNA is subcloned into the expression plasmid phD (Trans-activated expression of fully gamma-carboxylated recombinant human protein C, an antithrombotic factor. Grinnell, B. W., Berg, D. T., Walls, J. and Yan, S. B. Bio/Technology 5: 1189-1192 (1987)). This plasmid DNA is transfected into 293HEK cells and selected with 200 μg/mL Hygromycin.

Cells are stimulated with the homodimeric protein of interest, and the cAMP generated within the cell is quantitated using an Amplified Luminescent Proximity Homogeneous Assay (Alpha Screen) from Perkin Elmer (6760625R). Briefly, cAMP induced within the cell competes for binding of biotinylated cAMP from the kit to a coated anti-cAMP antibody Acceptor bead and a strepavidin coated Donor bead. As the cAMP level within the cell increases, a disruption of the Acceptor bead-biotinylated cAMP-Donor bead complex occurs and decreases the signal which is observed.

The hGLP-1R-HEK293 cells are harvested from sub-confluent tissue culture dishes with Enzyme-Free Cell Dissociation Solution, (Specialty Media 5-004-B). The cells are pelleted at low speed and washed 3 times with assay buffer [25 mM HEPES in HBSS-with Mg and Ca (GIBCO, 14025-092) with 0.1% Fatty Acid Free BSA] then diluted to a final concentration of 125,000 cells per mL. Biotinylated cAMP from the Alpha Screen kit is added to the diluted cells at a final concentration of 1 unit/0.04 mL. A phosphodiesterase inhibitor, IBMX (250 mM in DMSO), is also added to the diluted cells to a final concentration of 500 μM. GLP-1 is stored at 1 mg/mL in PBS as frozen aliquots at −80° C. The GLP-1, cAMP standard, and the homodimeric protein of interest are serially diluted into Assay buffer to a 6× final concentration. The functional assay is performed in 96 well, low volume, white, polystyrene Costar Plates (3688). The reaction starts by adding 0.01 mL of the diluted homodimeric protein, GLP-1, or cAMP into 0.04 mL of the cell mixture. After 1 hr at room temperature, the reaction is stopped by the addition of 0.03 mL of Lysis Buffer [10 mM HEPES, pH 7.4, 1% NP40, and 0.01% fatty acid free BSA containing 1 unit each/0.03 mL of Acceptor and Donor beads from the Alpha Screen Kit]. Addition of the lysis buffer is performed in the dark to prevent bleaching of the detection beads. The plates are wrapped in foil, gently shaken for 1 min then left to equilibrate overnight at room temperature. The plates are read on a Perkin-Elmer Envision instrument. The Alpha screen units are converted into pmoles cAMP generated per well based upon the cAMP standard curve. The pmoles cAMP generated in each well is converted to a percent of the maximal response observed with the GLP-1 control. An EC₅₀ value is derived by non-linear regression analysis using the percent maximal response vs. the concentration of peptide added.

The average in vitro potency (EC₅₀) of the homodimeric protein wherein the amino acid sequence of each polypeptide of said protein is SEQ ID NO: 1 at hGLP-1R is 0.067±0.011 nM with 95% confidence interval of 0.033 to 0.136.

### EXAMPLE 5

### Physical Stability

To determine the physical stability of the homodimeric protein of the present invention, the homodimeric protein is dialyzed and prepared at 1-2 mg/mL in 10 mM Citrate pH 7, 150 mM NaCl and analyzed by SEC to determine the % HMW (Table 1: “Initial”).

The SEC separation method is performed on a Tosoh Bioscience 3000SWXL, 5 micron column with dimensions 30 cm×0.78 cm. Mobile phase is 0.01 M sodium citrate 150 mM NaCl, pH 7 at a flow rate of 0.5 mL/minute. Initial low concentration samples are applied as 10 mcL injections and monitored at an absorbance wavelength of 214 nm, whereas the 50 mg/mL samples are applied as 1 mcL injections and monitored at 280 nm.

The homodimeric protein is concentrated to 50 mg/mL and analyzed again (t=0). The % HMW for the homodimeric protein wherein the amino acid sequence of each polypeptide of said protein is SEQ ID NO: 1 increased from 0.9% to 1.4% upon concentration. Thus, the homodimeric protein wherein the amino acid sequence of each polypeptide of said protein is SEQ ID NO: 1 has low initial % HMW and low % HMW when the protein is formulated at 50 mg/mL.

The 50 mg/mL formulations are incubated for 4 weeks at 4° C., 25° C., and 40° C. to assess longer-term stability under stress conditions. As shown in Table 1, the % HMW is determined again at 4 weeks time (t=4 weeks). The % HMW for the homodimeric protein wherein the amino acid sequence of each polypeptide of said protein is SEQ ID NO: 1 increased from 1.9% to 4.9% at 40° C. After 4 weeks at 25° C., levels of % HMW were 3.3% for the homodimeric protein wherein the amino acid sequence of each polypeptide of said protein is SEQ ID NO: 1.

TABLE 1
Physical Stability
Homodimeric protein wherein the amino acid sequence
of each polypeptide of said protein is SEQ ID NO: 1

| | | 50 mg/mL | 50 mg/mL |
|---|---|---|---|
| | | % HMW | % HMW |
| | Initial | (t = 0) | (t = 4 weeks) |
| 10 mM Citrate pH 7, 150 mM NaCl | 1.4% | 1.9% | |
| 4° C. | | | 2.0% |
| 25° C. | | | 3.3% |
| 40° C. | | | 4.9% |

### EXAMPLE 6

### Self-Association at High Concentration

To test for the propensity of the homodimeric protein of the present invention to self-associate, protein is dialyzed into the buffers listed in Table 2 and analyzed by size exclusion chromatography (SEC) to determine the % high molecular weight (% HMW) of a 1.0 mg/mL and 75 mg/ml solution. % HMW is an indicator of protein aggregation and self-association.

The SEC separation method is performed on a Tosoh Bioscience 3000SWXL, 5 micron column with dimensions 30 cm×0.78 cm. Mobile phase is 0.01 M sodium citrate, 150 mM NaCl, pH 7 at a flow rate of 0.5 mL/minute. 1.0 mg/mL samples are applied as 10 mcL injections and monitored at an absorbance wavelength of 214 nm, whereas 75 mg/mL samples are applied as 1 mcL injections and monitored at 280 nm.

TABLE 2
Self-Association
Homodimeric protein wherein the amino acid sequence
of each polypeptide of said protein is SEQ ID NO: 1

| | | | 10 mM Citrate, |
|---|---|---|---|
| Buffer | 10 mM Citrate, | 10 mM Citrate, | 50 mM NaCl, |
| Composition | 150 mM NaCl | 50 mM NaCl | 100 mM arginine |
| HMW (%) | 1.4% | 1.3% | 1% |
| 1 mg/mL | | | |
| HMW (%) | 1.9% | 1.8% | 1.2% |
| 75 mg/mL | | | |

Table 2 illustrates 1.0-1.4% HMW for the homodimeric protein wherein the amino acid sequence of each polypeptide of said protein is SEQ ID NO: 1. Samples are then concentrated to 75 mg/mL to simulate a high concentration formulation and analyzed again by SEC to determine the % HMW. The homodimeric protein wherein the amino acid sequence of each polypeptide of said protein is SEQ ID NO: 1 contained only 1.2-1.9% HMW at 75 mg/mL.

### EXAMPLE 7

### Glucose Lowering in ob/ob Mouse Model

Male ob/ob mice and age-matched ob/m (lean) controls are 7 weeks of age upon arrival and 8-9 weeks of age at initiation of treatment. Upon arrival, all mice are single housed and allowed to acclimate for 1-2 weeks before the start of treatment. The mice are fed Purina Rodent Chow 5015 and given house water from an auto-water apparatus ad libitum. The mice are housed in 12-hour light/dark cycle with ambient temperature set at 75° F. One to two days prior to initiation of treatment, blood samples are collected via tail bleed. Blood glucose levels are measured using an Accu-Check Avivia blood glucose meter (Roche) and serum samples are collected for the assay of insulin using the Meso Scale mouse/rat insulin assay kit. On the day of treatment initiation (day 0), the mice are sorted into groups based on the pretreatment body weight, blood glucose, and serum insulin (BRAT sorting software). On day 0 and day 3, mice are dosed SQ with 0.3 to 30 nmol/kg of the homodimeric protein wherein the amino acid sequence of each polypeptide of said protein is SEQ ID NO: 1, in a volume of 10 mL/kg. Dosing vehicle is sterile PBS (HyClone DPBS/Modified—Calcium—Magnesium) containing 0.03% mouse serum albumin (MSA; Sigma A3139). Blood glucose is measured daily for 7 days and the AUC is determined. ED₅₀ calculations for the glucose lowering are based on the AUC. Liver homogenates collected at the time of sacrifice and liver triglycerides are measured on the Hitachi Modular P clinical analyzer.

On day 7, vehicle treated mice were hyperglycemic with mean blood glucose levels measured at 387±63.0 mg/dl (mean±SEM), while ob/m lean control mice had blood glucose levels of 162±9.0 mg/dl (mean±SEM). The homodimeric protein wherein the amino acid sequence of each polypeptide of said protein is SEQ ID NO: 1 lowered blood glucose to levels comparable to the ob/m lean controls. The glucose AUC ED₅₀ of the homodimeric protein wherein the amino acid sequence of each polypeptide of said protein is SEQ ID NO: 1 was 2.95 nmol/kg (95% confidence interval=2.06-4.23).

### EXAMPLE 8

### Diet-Induced Obesity (DIO) Mouse Model

Homodimeric protein wherein the amino acid sequence of each polypeptide of said protein is SEQ ID NO: 1 is dosed in C57B16 diet-induced obese (DIO) mice. These animals, although not diabetic, display insulin resistance, dyslipidemia, and hepatic steatosis, all characteristics of metabolic syndrome, after being placed on a high fat (60% Kcal from fat) diet for 12 weeks.

Three to four month old male diet-induced obese (DIO) C57/B16 male mice weighing 37-45 g are individually housed in a temperature-controlled (24° C.) facility with a 12 hour light/dark cycle (lights on 22:00), and had free access to food and water. After 2 weeks acclimation to the facility, mice are randomized to treatment groups (n=5/group) based on body weight and fat mass, so each group has similar starting mean body weight and fat mass.

Vehicle or the homodimeric protein wherein the amino acid sequence of each polypeptide of said protein is SEQ ID NO: 1 (dose range 1 to 30 nmol/kg) dissolved in vehicle (phosphate buffered saline, Hyclone, ThermoScientific) are administered by SQ injection to ad libitum fed DIO mice 30-90 minutes prior to the onset of the dark cycle every three days for 15 days. SQ injections are made on Day 1, 4, 7, 10, and 13. Daily body weight and food intake are measured throughout the study. Absolute changes in body weight are calculated by subtracting the body weight of the same animal prior to the first injection of molecule. On days 1 and 15, total fat mass is measured by nuclear magnetic resonance (NMR) using an Echo Medical System (Houston, Tex.) instrument.

Following body composition measurements, animals are sacrificed and livers removed and frozen. Liver triglycerides are determined from homogenates made from livers collected at sacrifice and measured on the Hitachi Modular P clinical analyzer. Statistical comparisons between groups are done using one-way ANOVA followed by Dunnett's multiple comparison test. The ED₅₀ for weight loss lowering are determined in GraphPad Prism using the non-linear fit tool.

The homodimeric protein wherein the amino acid sequence of each polypeptide of said protein is SEQ ID NO: 1 dose-dependently reduced body weight and fat mass. The ED₅₀ of the homodimeric protein wherein the amino acid sequence of each polypeptide of said protein is SEQ ID NO: 1 in percent body weight loss was 7.577 nmol/kg. Reduced body weight was primarily due to reduction in fat mass. However, due to extensive weight loss in a short period of time, significant total water loss was also observed (see Table 3). The homodimeric protein wherein the amino acid sequence of each polypeptide of said protein is SEQ ID NO: 1 also caused 70% reduction in plasma triglycerides and 95% in liver triglycerides at the end of the study.

TABLE 3
The effect of the homodimeric protein wherein the amino acid sequence of each
polypeptide of said protein is SEQ ID NO: 1 on body composition in DIO mouse
(negative numbers indicate the amount of actual loss as grams)

| | BW Change (g) | Fat Mass Change (g) | Total Water (g) |
|---|---|---|---|
| Treatment | (mean ± SEM) | (mean ± SEM) | (mean ± SEM) |
| Vehicle | −0.82 ± 0.59 g | 0.00 ± 0.54 g | −0.32 ± 0.34 |
| (10 mL/kg, SC) | | | |
| Homodimeric protein | −0.70 ± 0.34 g | −0.19 ± 0.23 g | −0.37 ± 0.17 g |
| wherein the amino acid | | | |
| sequence of each | | | |
| polypeptide of said | | | |
| protein is SEQ ID NO: 1 | | | |
| (1 nmol/kg, SC) | | | |
| Homodimeric protein | −4.78 ± 0.50 g | −3.11 ± 0.44 g | −1.11 ± 0.12 g |
| wherein the amino acid | | | |
| sequence of each | | | |
| polypeptide of said | | | |
| protein is SEQ ID NO: 1 | | | |
| (3 nmol/kg, SC) | | | |
| Homodimeric protein | −11.18 ± 0.65 g | −8.35 ± 0.44 g | −1.96 ± 0.20 g |
| wherein the amino acid | | | |
| sequence of each | | | |
| polypeptide of said | | | |
| protein is SEQ ID NO: 1 | | | |
| (10 nmol/kg, SC) | | | |
| Homodimeric protein | −14.66 ± 0.76 | −11.54 ± 0.44 g | −1.92 ± 0.34 |
| wherein the amino acid | | | |
| sequence of each | | | |
| polypeptide of said | | | |
| protein is SEQ ID NO: 1 | | | |
| (30 nmol/kg, SC) | | | |

### Sequences

SEQ ID NO: 1 -
Homodimeric protein
HGEGTFTSDVSSYLEEQAAKEFIAWLVAGGGGGGGSGGGGSGGGGSESKY
GPPCPPCPAPEAAGGPSVFLFPPKPKDTLMISRTPEVTCVVVDVSCEDPE
VQFNWYVDGVEVHNAKTKPREEQFNSTYRVVSVLTVLHQDWLNGKEYKCK
VSNKGLPSSIEKTISKAKGQPREPQVYTLPPSCEEMTKNQVSLTCLVKGF
YPSDIAVEWESNGQPENNYKTTPPVLDSDGSFFLYSRLTVDKSRWQEGNV
FSCSVMHEALHNHYTQKSLSLSLGGGGGSGGGGSGGGGSAHPIPDSSPLL
QFGGQVRQRYLYTDDAQQTECHLEIREDGTVGCAADQSPESLLQLKALKP
GVIQILGVKTSRFLCQRPDGALYGSLHFDPEACSFREDLKEDGYNVYQSE
AHGLPLHLPGDKSPHRKPAPRGPARFLPLPGLPPALPEPPGILAPQPPDV
GSSDPLRLVEPSCLRSPSFE
SEQ ID NO: 2 -
(DNA) of the Homodimeric protein
GAGTCCAAATATGGTCCCCCATGCCCACCCTGCCCAGCACCTGAGGCCGC
CGGGGGACCATCAGTCTTCCTGTTCCCCCCAAAACCCAAGGACACTCTCA
TGATCTCCCGGACCCCTGAGGTCACGTGCGTGGTGGTGGACGTGAGCCAG
GAAGACCCCGAGGTCCAGTTCAACTGGTACGTGGATGGCGTGGAGGTGCA
TAATGCCAAGACAAAGCCGCGGGAGGAGCAGTTCAACAGCACGTACCGTG
TGGTCAGCGTCCTCACCGTCCTGCACCAGGACTGGCTGAACGGCAAGGAG
TACAAGTGCAAGGTCTCCAACAAAGGCCTCCCGTCCTCCATCGAGAAAAC
CATCTCCAAAGCCAAAGGGCAGCCCCGAGAGCCACAGGTGTACACCCTGC
CCCCATCCCAGGAGGAGATGACCAAGAACCAGGTCAGCCTGACCTGCCTG
GTCAAAGGCTTCTACCCCAGCGACATCGCCGTGGAGTGGGAAAGCAATGG
GCAGCCGGAGAACAACTACAAGACCACGCCTCCCGTGCTGGACTCCGACG
GCTCCTTCTTCCTCTACAGCAGGCTAACCGTGGACAAGAGCAGGTGGCAG
GAGGGGAATGTCTTCTCATGCTCCGTGATGCATGAGGCTCTGCACAACCA
CTACACACAGAAGAGCCTCTCCCTGTCTCTGGGTGGTGGTGGTGGCTCCG
GAGGCGGCGGCTCTGGTGGCGGTGGCAGCGCTCACCCCATCCCTGACTCC
AGTCCTCTCCTGCAATTCGGGGGCCAAGTCCGGCAGCGGTACCTGTACAC
CGACGACGCCCAGCAGACCGAGTGCCACCTGGAAATCCGGGAGGACGGCA
CCGTGGGCTGTGCCGCCGACCAGTCCCCTGAGTCCCTGCTGCAGCTGAAG
GCCCTGAAGCCTGGCGTGATCCAGATCCTGGGCGTGAAAACCTCCCGGTT
CCTGTGCCAGAGGCCTGATGGCGCCCTGTACGGCTCCCTGCACTTCGACC
CTGAGGCCTGCTCCTTCCGGGAGGACCTGAAGGAAGATGGCTACAACGTG
TACCAGTCCGAGGCTCACGGCCTGCCTCTGCATCTGCCTGGCGACAAGTC
CCCCCACCGGAAGCCTGCTCCTAGGGGCCCTGCCAGATTCCTGCCACTGC
CTGGCCTGCCTCCAGCTCTGCCTGAGCCTCCTGGCATCCTGGCCCCTCAG
CCTCCAGACGTGGGCTCCTCCGACCCTCTGCGGCTGGTCGAGCCTTCCCA
GCTGCGGAGCCCTAGCTTCGAG
SEQ ID NO: 3 -
Human transferrin (hTrf) Signal Peptide
MRLAVGALLVCAVLGLCLA
SEQ ID NO: 4 -
Human fibroblast growth factor binding protein-1
(hFGFP-1)
Signal Peptide
METDTLLLWVLLLWVPGSTG

We claim:

1. A homodimeric protein wherein the amino acid sequence of each polypeptide of said protein is
(SEQ ID NO: 1)
HGEGTFTSDVSSYLEEQAAKEFIAWLVAGGGGGGGSGGGGSGGGGSESK
YGPPCPPCPAPEAAGGPSVFLEPPKPKDTLMISRTPEVTCVVVDVSCEDP
EVQFNWYVDGVEVHNAKTKPREEQFNSTYRVVSVLTVLHQDWLNGKEYK
CKVSNKGLPSSIEKTISKAKGQPREPQVYTLPPSCEEMTKNQVSLTCLVK
GFYPSDIAVEWESNGQPENNYKTTPPVLDSDGSFFLYSRLTVDKSRWQEG
NVFSCSVMHEALHNHYTQKSLSLSLGGGGGSGGGGSGGGGSAHPIPDSSP
LLQFGGQVRQRYLYTDDAQQTECHLEIREDGTVGCAADQSPESLLQLKAL
KPGVIQILGVKTSRFLCQRPDGALYGSLHFDPEACSFREDLKEDGYNVYQ
SEAHGLPLHLPGDKSPHRKPAPRGPARFLPLPGLPPALPEPPGILAPQPP
DVGSSDPLRLVEPSCLRSPSFE.

2. A DNA molecule encoding a polypeptide, wherein the amino acid sequence of the polypeptide is SEQ ID NO: 1.

3. A mammalian host cell transformed with a DNA molecule of claim 2, which cell is capable of expressing a homodimeric protein wherein the amino acid sequence of each polypeptide of said protein is SEQ ID NO: 1.

4. A process for producing a homodimeric protein wherein the amino acid sequence of each polypeptide of said protein is SEQ ID NO: 1, said process comprising the steps of:
i) cultivating a mammalian host cell comprising a polynucleotide encoding the polypeptide having the amino acid sequence of SEQ ID NO: 1 under conditions such that said polypeptide sequence is expressed; and
ii) recovering from said host cell a homodimeric protein, wherein the amino acid sequence of each polypeptide of said homodimeric protein is SEQ ID NO: 1.

5. A homodimeric protein produced by the process of claim 4.

6. A pharmaceutical composition comprising the homodimeric protein of claim 1, and at least one pharmaceutically acceptable carrier, diluent, or excipient.

7. A method for treating type 2 diabetes, obesity, dyslipidemia, and/or metabolic syndrome comprising administering a homodimeric protein of claim 1 to a patient in need thereof.

8. A pharmaceutical composition comprising the homodimeric protein of claim 5, and at least one pharmaceutically acceptable carrier, diluent, or excipient.

9. A method for treating type 2 diabetes, obesity, dyslipidemia, and/or metabolic syndrome comprising administering a homodimeric protein of claim 5 to a patient in need thereof.


SEQUENCE LISTING (4 sequences)

SEQ ID NO: 1 - protein, 470 aa, Artificial Sequence
  Synthetic Construct
HGEGTFTSDVSSYLEEQAAKEFIAWLVAGGGGGGGSGGGGSGGGGSESKYGPPCPPCPAPEAAGGPSVFLFPPKPKDTLMISRTPEVTCVVVDVSCEDPEVQFNWYVDGVEVHNAKTKPREEQFNSTYRVVSVLTVLHQDWLNGKEYKCKVSNKGLPSSIEKTISKAKGQPREPQVYTLPPSCEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSAHPIPDSSPLLQFGGQVRQRYLYTDDAQQTECHLEIREDGTVGCAADQSPESLLQLKALKPGVIQILGVKTSRFLCQRPDGALYGSLHFDPEACSFREDLKEDGYNVYQSEAHGLPLHLPGDKSPHRKPAPRGPARFLPLPGLPPALPEPPGILAPQPPDVGSSDPLRLVEPSCLRSPSFE

SEQ ID NO: 2 - DNA, 1272 bp, Artificial Sequence
  Synthetic Construct
gagtccaaatatggtcccccatgcccaccctgcccagcacctgaggccgccgggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggaaagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtggtggtggtggctccggaggcggcggctctggtggcggtggcagcgctcaccccatccctgactccagtcctctcctgcaattcgggggccaagtccggcagcggtacctgtacaccgacgacgcccagcagaccgagtgccacctggaaatccgggaggacggcaccgtgggctgtgccgccgaccagtcccctgagtccctgctgcagctgaaggccctgaagcctggcgtgatccagatcctgggcgtgaaaacctcccggttcctgtgccagaggcctgatggcgccctgtacggctccctgcacttcgaccctgaggcctgctccttccgggaggacctgaaggaagatggctacaacgtgtaccagtccgaggctcacggcctgcctctgcatctgcctggcgacaagtccccccaccggaagcctgctcctaggggccctgccagattcctgccactgcctggcctgcctccagctctgcctgagcctcctggcatcctggcccctcagcctccagacgtgggctcctccgaccctctgcggctggtcgagccttcccagctgcggagccctagcttcgag

SEQ ID NO: 3 - protein, 19 aa, Artificial Sequence
  Synthetic Construct
MRLAVGALLVCAVLGLCLA

SEQ ID NO: 4 - protein, 20 aa, Artificial Sequence
  Synthetic Construct
METDTLLLWVLLLWVPGSTG